(19) United States
(12) Patent Application Publication
(10) Pub. No.: US 2009/0324708 A1
(43) Pub. Date: Dec. 31, 2009

(54) COMPOUND HAVING INHIBITORY ACTIVITY ON A RHO-GTPASE CELL PROTEIN, A PROCESS FOR OBTAINING THE SAME, PHARMACEUTICAL COMPOSITIONS COMPRISING THEREOF AND A METHOD FOR THE TREATMENT OF RHO-GTPASE CELL PROTEIN-MEDIATED CONDITION

(75) Inventors: Daniel Fernando Alonso, Provincia de Buenos Aires (AR); Daniel Eduardo Gomez, Provincia de Buenos Aires (AR); Luis Emilio Iglesias, Ciudad de Buenos Aires (AR); Maria Alejandra Zinni, Provincia de Buenos Aires (AR); Adolfo Marcelo Iribarren, Provincia de Buenos Aires (AR); Pablo Lorenzano Menna, Provincia de Buenos Aires (AR)

Correspondence Address: SUGHRUE MION, PLLC, 2100 PENNSYLVANIA AVENUE, N.W., SUITE 800, WASHINGTON, DC, 20037, US

(73) Assignees: Universidad Nacional De Quilmes, Provincia de Buenos Aires (AR); Consejo Nacional De Investigaciones Cientificas Y Tecnicas, Cludad de Buenos Aires (AR)

(21) Appl. No.: 12/513,815

(22) PCT Filed: Nov. 5, 2007
(86) PCT No.: PCT/EP2007/061887

§ 371 Date: Jun. 15, 2009

(30) Foreign Application Priority Data
Nov. 6, 2006 (AR) P20060104854

(51) Int. Cl. A61K 9/127; C07H 19/16; A61K 31/7076; A61K 9/14; A61P 35/00

(52) U.S. Cl. 424/450; 536/27.22; 514/45; 424/489

(57) ABSTRACT

The present invention relates to a compound having inhibitory activity on a Rho-GTPase cell protein, the compound having the formula I (Formula I) wherein A is selected from N and N—H, R₁ is selected from H and NHR₃, R₂ is selected from NHR₄, OR₄, O and halogen, R₃ is selected from H and —COR₅, R₄ is selected from H, a C₁-C₆ alkyl and a substituted or unsubstituted phenyl, R₅ is selected from a C₁-C₁₂ alkyl and a substituted or unsubstituted phenyl, R₆ is selected from H, —COR₅, —CO₂R₅, —PR₇R₈ and —PR₇R₈OPR₇R₈R₈R₇, R₇ is selected from O and S, R₈ is selected from H, OR₄ and OSATE (—OCH₂CH₂SCOR₅), and wherein each represents a single bond or a double bond, provided that when one of them is a double bond the other one is a single bond, and pharmaceutically acceptable salts and derivatives thereof. In particular, the compounds of the invention may be used as antitumor agents the action of which interfere with the signaling pathways normally involved in tumor development processes. The invention also relates to processes for obtaining of compounds having inhibitory activity of a Rho-GTPase cell protein, to the pharmaceutical compositions thereof and to the therapeutic methods comprising the use of said compounds and compositions.

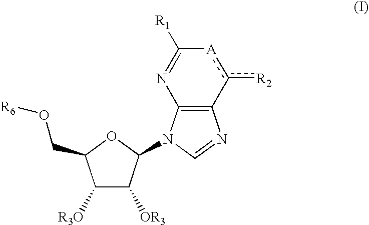

The present invention relates to a compound having inhibitory activity on a Rho-GTPase cell protein, the compound having the general formula I

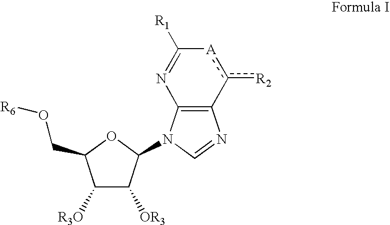

wherein
A is selected from N and N—H
R₁ is selected from H and NHR₃,
R₂ is selected from NHR₄, OR₄, O and halogen,
R₃ is selected from H and —COR₅,
R₄ is selected from H, C₁-C₆ alkyl and a substituted or unsubstituted phenyl,
R₅ is selected from C₁-C₁₂ alkyl and a substituted or unsubstituted phenyl,
R₆ is selected from H, —COR₅, —CO₂R₅, —PR₇R₈ and —PR₇R₈OPR₇R₈R₈R₇
R₇ is selected from O and S,

R₈ is selected from H, OR₄ and OSATE (—OCH₂CH₂SCOR₅), and where each [symbol] represents a single bond or a double bond, provided that when one of them is a double bond, the other one is a single bond, and pharmaceutically acceptable salts and derivatives thereof.

Particularly, the inventive compounds may be used as antitumor agents the action of which interferes with signaling pathways normally involved in tumor development processes. Present invention relates also to processes for obtaining compounds with inhibitory activity on Rho-GTPase cell proteins, to pharmaceutical compositions comprising thereof and to therapeutic methods comprising the use of said compounds and compositions.

### BACKGROUND OF THE INVENTION

Rho proteins constitute a subfamily from the Ras GTPases super-family. More than fifty members of said super-family are grouped, upon their amino acid composition homology, in the following subfamilies: Ras, Rho, Rab, Arf, Ran and Rad. These proteins have similar molecular weights (20-25 kDa) and are generally named small GTPases or Small GTP-binding proteins, thereby being different from the remaining proteins with GTPase activity, such as the heterotrimeric G proteins.

Mammal Rho subfamily is constituted by several members: Rho A, B, C, D and E; Rac 1, 2 and 3; Cdc 42, TC 10 and others. The structure of these proteins is very similar (40 to 95% amino acidic composition identity) being very similar in size (190-195 amino acids).

One of the main features of Rho proteins, like all G proteins, is their capability of binding guanine nucleotides, and cycling from an inactive state linked to GDP to an active state linked to GTP. These proteins, like Ras proteins, show endogen GTPase activity, being able to hydrolyze GTP in GDP by a Mg²⁺-dependent process. It is assumed that Ras super-family proteins interact with their effectors when bound to GTP, which, at the same time, interact with other proteins thus resulting in a signaling cascade.

The Rho GTPases have functional domains in common, similar to those of Ras. Such domains consist of four regions that take part in the binding and hydrolysis of guanine nucleotide (G1, G3, G4 and G5), a terminal sequence CXXX (C being cystein and X any other amino acid), and a region G2 involved in the interaction with effector molecules (Haeusler L C H, Blumenstein L, Stege P, Dvorsky R, Ahmadian M R. Comparative Functional analysis of the Rac GTPases. FEBS Letters 2003, 555: 556-560).

Rho GTPases regulate cell morphology and actin cytoskeleton reorganization. Thus, Rho can be activated by extracellular ligands such as lysophosphatidic acid and regulate stress fiber formation. Rac is activated by several growth factors, such as platelet derived growth factor (PDGF), EGF or Insulin, giving rise to lamellipodia formation and membrane ruffling. Besides, Cdc42 activation produces filopodiae (Sahai E, Marshall C J. Rho-GTPases and cancer. Nature 2002, 2: 133-142; Takai Y, Sasaki T, Matozaki T. Small GTP-binding proteins. Physiological Reviews 2001, 81: 153-208; Kjoller L, Hall A. Signaling to Rho GTPases. Experimental Cell Research 1999, 253: 166-179; Symons M, Derry J, Karlak B, Jiang S, Limahieu L, McCormick F. Wiskott-Aldrich protein, a novel effector of the GTPases Cdc42HS is implicated in actin polymerization. Cell 1996, 84: 723-734).

The morphologic changes induced by activated forms of this GTPases family share many aspects: increase in actin polymerization, integrin grouping, and assembly of cytoskeleton protein complexes (focal contacts). Focal contacts play a relevant role in the signaling transduction mechanisms. Integrin-mediated cellular adhesion triggers tyrosine phosphorylation, ion flow and lipid metabolism, that ultimately, jointly or individually, affect the gene expression, the cell cycle progression and apoptosis processes (Sahai E, Marshall C J. Rho-GTPases and cancer. Nature 2002, 2: 133-142; Symons M, Derry J, Karlak B, Jiang S, Limahieu L, McCormick F. Wiskott-Aldrich protein, a novel effector of the GTPases Cdc42HS is implicated in actin polymerization. Cell 1996, 84: 723-734; Schwartz M A and Shattil S J. Signaling networks linking integrins and Rho family GTPases. Science 2000 25: 388-391).

In addition to actions on the cytoskeleton, Rho, Rac and Cdc42 proteins, regulate a wide range of cell functions, thus mediating transcriptional regulation of certain genes. For instance, Rac stimulates the Jun kinase cascade (JNK), thereby transmitting information to the nucleus, to regulate the expression of a relevant number of genes (Coso O A, Chiariello M, Yu J, Teramoto H, Crespo P, Xu N, Miki T, Gutkind J S. The small GTP-binding proteins Rac and Cdc42 regulate the activity of the JNK (SAPK) signaling pathway. Cell 1995, 81: 1137-1146; Kaempchen K, Mielke K, Utermark T, Langmesser S, Hanemann C O. Upregulation of the Rac1/JNK signaling pathway in primary human schwannoma cells. Hum Mol Genet. 2003, 12(11):1211-21; Kam A Y, Chan A S, Wong Y H. Rac and Cdc42-dependent regulation of c-Jun N-terminal kinases by the delta-opioid receptor. J Neurochem 2003, 84(3):503-13; Kam A Y, Chan A S, Wong Y H. Rac and Cdc42-dependent regulation of c-Jun N-terminal kinases by the delta-opioid receptor. J Neurochem 2003, 84(3):503-13). Additionally, among other functions, they participate in cell cycle and cell division regulation, being also involved in secretion, endocytosis, phagocytosis, membrane traffic and apoptosis.

The wide range of biological functions of the different members of Rho-GTPases family is due to their binding to different cell effectors. Subsequently, in this sense, some of the more representative ones, as to their participation in tumorigenesis associated with Rho-GTPases, will be mentioned.

Firstly, it can be mentioned the WASP (Wiscott-Aldrich syndrome protein) protein family. This protein was originally discovered as a protein possessing a Cdc42 specific CRIB domain (Cdc42/Rac interactive binding), not having a defined catalytic activity (Symons M, Derry J, Karlak B, Jiang S, Limahieu L, McCormick F. Wiskott-Aldrich protein, a novel effector of the GTPases Cdc42HS is implicated in actin polymerization. Cell 1996, 84: 723-734; Derry J, Ochs J, Francke U. Isolation of a novel gene mutated in Wiskott-Aldrich syndrome. Cell 1996, 78: 635-644). This protein binds to Cdc42 active conformation, and it is involved in actin polymerization by means of Arp2/3 complex and the necessary assembly/disassembly of the podosome necessary for cell migration (Aspenstrom P, Lindberg L, Hall A. Two GTPases Cdc42 and Rac bind directly to a protein implicated in the immunodeficiency disorder Wiskott-Aldrich syndrome. Curr Biol 1996, 6: 70-75; Linder S, Nelson D, Weiss M, Aepfelbacher M. Wiskott-Aldrich syndrome protein regulates podosomes in primary human macrophages. Proc. Natl. Acad Sci USA 96 1999, 9648-9653; Yarar D, To W, Abo A, Welch M D. The Wiskott-Aldrich syndrome protein directs actin based motility by stimulating actin nucleation with the Arp2/3 complex. Curr. Biol 1999, 9: 555-558). A second member of the WASP Family, named WAVE, was cloned in order to study its relationship with Rho-GTPases. WAVE selectively binds to Rac1, but not to Cdc42 (Miki H, Suetsugu S, Takenawa T. WAVE a novel WASP family protein involved in actin reorganization induced by Rac. Eur. Mol. Biol. Org. J 1998, 17: 6932-6941). The WAVE activation after its binding to Rac1, causes aggregation of actin filaments and its binding to Porphiline, so as to mediate Lamellipodia formation and membrane ruffling (Miki H, Suetsugu S, Takenawa T. WAVE a novel WASP family protein involved in actin reorganization induced by Rac. Eur. Mol. Biol. Org. J 1998, 17: 6932-6941; Aelst L V, Joneson T, Bar-Sagi D. Identification of a novel Rac1-interacting protein involved in membrane ruffling. Eur. Mol. Biol. Org. J 1996, 15: 3778-3786). WAVE hyperphosphorylation was found in several tumoral cell lines, that was correlated with the increasing of membrane ruffles, which are necessary for tumoral migration (Miki H, Suetsugu S, Takenawa T. WAVE a novel WASP family protein involved in actin reorganization induced by Rac. Eur. Mol. Biol. Org. J 1998, 17: 6932-6941). In addition, a genic translocation causing WAVE3 gene deactivation in children with ganglioneuroblastoma was recently detected (Sossey-Alaoui K, Su G, Malaj E, Roe B, Cowell J K. WAVE3 an actin-polymerization gene is truncated and inactivated as a result of a constitutional t (1;13) (q21;q12) chromosome translocation in a patient with ganglioneuroblastoma. Oncogene 2002, 21: 5967-5974). This suggests that WAVE may act as tumoral suppressor.

Another important effector of Rho family is IQGAP, which interacts with Rac1 and Cdc42, and it is located on the membrane ruffles and in cell-cell contacts (Kuroda S, Fucata M, Nakagawa M, Kaibuchi K. Cdc42, Rac and their effector IAGAP as molecular switches for cadherin-mediated cell-cell adhesion. Biocehm. Biophys. Res. Commun 1999, 262: 1-6; Kuroda S, Fukata M, Nakagawa M, Fujii K, Nakamura T, Ookubo T. Role of IQGAP1, a target of the small GTPase Cdc42 and Rac1, in regulation of E. cadherin-mediated cell-cell adhesion. Science 1998, 281: 832-835). IQGAP1 accumulates in cell-cell binding sites by an E-cadherin/β-cathenin union dependent mechanism, and together with MRCK, modulate formation of adhesive Rac/Cdc42-dependant bindings (Kuroda S, Fukata M, Nakagawa M, Fujii K, Nakamura T, Ookubo T. Role of IQGAP1, a target of the small GTPase Cdc42 and Rac1, in regulation of E. cadherin-mediated cell-cell adhesion. Science 1998, 281: 832-835). Though IQGAP binds only to Rac and Cdc42, Rhoa can regulate E-cadherin activity, possibly by a cytoskeleton reorganization-dependant mechanism. It is not clear enough the Rac and Cdc42 roles in the regulation of cadherin-dependant unions. In this sense, though Rac and Cdc42 can induce formation of adhesive bindings by IQGAP inhibition, according to cell circumstances, both are known inductors of cell migration (Schmit A A P, Govec E E, Bottner B, Aelst A V. Rho-GTPases: signaling, migration and invasion. Exp. Cell Res 2002, 261: 1-12).

PAK proteins (p21—Activated kinase) are a third group of effectors pertaining to small GTPases family. PAK proteins are serine and threonine kinases that activate after binding with Rac and Cdc42 active conformation (Teo M, Manser E, Lim L. Identification and molecular cloning of a p21Cdc42/Rac1 activated serine/threonine kinase that is rapidly activated by thrombin in platelets. J. Biol. Chem. 1995, 270: 26690-26697; Bagrodia S, Taylor S J, Jordon K A, Aelst L V, Cerione R. A novel regulator of p21-activated kinases. J. Biol. Chem. 1998, 273: 23633-23636). PAK kinases are involved in Rho-GTPases-mediated tumorigenesis. Assays carried out with murine fibroblasts, indicated that PAK activity was related to several aspects of tumoral biology. A PAK negative dominant expression prevents malign transformation of murine fibroblasts caused by oncogenes like Ras, Rac1, Cdc42 and Vav3 (Sachdev P R, Zeng L, Wang L H. Distinct role of phosphatidylinositol-3-kinase and Rho family GTPases in Vav3-induced cell transformation, cell motility and morphological changes. J. Biol. Chem. 2002, 277: 17638-17648; Tang Y, Chen Z, Ambrose D, Liu J, Gibbs J B, Chernoff J, Field J. Kinase-defective Pak1 mutants inhibit Ras transformation of Rat-1 fibroblasts. Mol. Cell. Biol 1998, 17: 4454-4464). PAK is capable of promoting cell proliferation and survival by phosphorylation and inhibition of Bad proapoptotic protein and caspase-3 trough an Akt-dependant mechanism (Tang Y, Zhou H, Chen A, Pittman R N, Field J. The Akt proto-oncogene link Ras to Pak and cell survival. J. Biol. Chem. 2000, 275: 9106-9109; Callow M G, Clairvoyant F, Zhu S, Schryver B, Whyte D B, Bischoff J R. Requirement for PAK-4 in the anchorage-independent growth of human cancer cell lines. J. Biol. Chem. 2002, 277: 550-558).

The necessity of PAK1 activity for tumoral growth and breast cancer metastasis has been reported. PAK1 inhibition in highly aggressive MDA-MB435 breast cancer cells prevented disassembly of focal unions and loss of stress fibers, thus resulting in cellular migration decrease (Vadlamudi R, Adam L, Wang R A, Mandal M, Nguyen D, Sahin A. Regulatable expression of p21-activated kinase-1 promotes anchorage independent growth and abnormal organization of mitotic spindles in human epithelial breast cancer cells. J. Biol. Chem. 2000, 275: 12041-12050). PAK1 expression in human breast tumors was correlated with the tumor grade, the expression being higher in the less differentiated ductal carcinoma (tumors of grade III) in regards to grade II or I tumors (Salh B, Marotta A, Wagey R, Sayed M, Pelech S. Dysregulation of phosphatidylinositol 3-kinase and downstream effectors in human breast cancer. Int. J. Cancer 2002, 98: 148-154).

In the last 5 years a lot of information related to the participation of the different members of the Rho-GTPases family in cellular processes associated to malignant transformation and other human pathologies, was accumulated (Boettner B and Van Aelst L: The role of Rho GTPases in disease development. Gene 2002, 286(2):155-74; Aznar S, Fernandez-Valeron P, Espina C, Lacal J C: Rho GTPases: potential candidates for anticancer therapy. Cancer Lett 2004, 206(2):181-91). Rho-GTPases mediate essential aspects of tumorigenesis induced by several oncogenes. This is the case of Ras, wherein the tumor growth and invasion of said oncogene transformed cells, depend on the Rho GTPases signaling pathways (Pruitt K and Der C J: Ras and Rho regulation of the cell cycle and oncogenesis. Cancer Lett 2001, 171(1):1-10; Qiu R G, Chen J, Kirn D, McCormick F, Symons M: An essential role for Rac in Ras transformation. Nature 1995, 374(6521):457-9). Besides, the signals mediated by Rho are necessary in the oncogenic phenotype induced by the tyrosine kinase-type receptors, like EGFR and IGFR, and receptors coupled to G proteins (McManus M J, Boerner J L, Danielsen A J, Wang Z, Matsumura F, Maihle N J: An oncogenic epidermal growth factor receptor signals via a p21-activated kinase-caldesmon-myosin phosphotyrosine complex. J Biol Chem 2000, 275(45):35328-34; Barone M V, Sepe L, Melillo R M, Mineo A, Santelli G, Monaco C, Castellone M D, Tramontano D, Fusco A and Santoro M: RET/PTC1 oncogene signaling in PC Cl 3 thyroid cells requires the small GTP-binding protein Rho. Oncogene 2001 20(48):6973-82; Royal I, Lamarche-Vane N, Lamorte L, Kaibuchi K, Park M: Activation of cdc42, rac, PAK, and rho-kinase in response to hepatocyte growth factor differentially regulates epithelial cell colony spreading and dissociation. Mol Biol Cell 2000, 11(5):1709-25).

Tumor cells, besides presenting proliferation deregulation, they present alterations in their morphological characteristics and, in the case of metastasis, they get the ability to pass through tissue barriers. It is apparent that Rho-GTPases play an important role in controlling cell morphology and motility. Cdc42 is necessary for filopodia formation, while Rac controls the formation process of lamellipodia, which are necessary structures for cell locomotion. RhoA is necessary for generating the cell contraction force and cell body motion in the migration process (Ridley A J: Rho: theme and variations. Curr. Biol 1996, 6: 1256-1264). In addition, these three proteins are involved in the formation of cell-matrix bonds mediated by integrins. Rho proteins are involved in the loss of cell polarization observed even in benign tumors, which is significant in epithelial-mesenchymal transition observed in more aggressive tumors.

The inhibition of Rac activity in MDCK cells (Madin-Darby Canine Kidney) transformed with Ras or stimulated with phorbol esters causes the inhibition of cell-cell bonds and the transition to mesenchymal phenotype with higher migratory capacity (Zondag G C, Evers E E, Ten Closter I P, Janseen L, vsan der Kammen R A and Collard J G: Oncogenic Ras downregulates Rac activity, which leads to increased Rho activity and epithelial-mesenchymal transition. J Cell Biol 2000 May 15; 149(4):775-82). Theses studies show that the decrease of certain Rho proteins can be a necessary alteration in tumor cells.

Although previous art publications are confusing about the role of Rho proteins in tumor progression, having found contradictory effects in different studies, said contradictions can be reconciled when considering that Rho proteins may have different functions in different stages of the tumor development.

The development of therapies with specific molecular targets is an area of great interest in present cancer research, having a huge potential in the search of a therapeutic benefit associated to a lower toxicity on normal cells. The GTPases family has the common characteristic of bonding guanosine nucleotides, which make the protein an interrupter, alternating between an active state bound to GTP and an inactive state when bound to GDP. This activation cycle is modulated by proteins which favor the inactive state (proteins GAP and GDI) and proteins favoring the active state (GEF or GRF).

On the aforesaid, the development of compounds capable of inhibiting Ras activity has been object of a great number of studies in laboratories all over the world. In this sense, the more studied agents have been the farnesyl-transferase inhibitors and mevalonate synthesis blockers. Both strategies are based on blocking the pos-translational addition of farnesyl or geranyl moieties, undergone by GTPases. These lipidic derivatives allow for the proper location of these proteins on the internal face of the membrane, where it bonds to its specific effectors. Currently, there are an important number of these agents in Phases 1 and 2 of clinical trials (W. S Kim et al. Invest New Drugs 19 (1) (2001) 81-3).

Present inventors have studied in detail the role of Rac1 GTPase in the molecular mechanisms related to tumor invasion and metastasis. Taking as a model a murine breast carcinoma highly invasive and metastatic, with high Rac1 expression levels, they have stably transfected the β2-chimerin active domain. This protein acts as a specific inhibitor of Rac activity. The heterologous overexpression of said protein resulted in a marked decrease of cell proliferation and migration, what produced a marked decrease of in vivo tumor invasion and metastasis.

On the basis of the results obtained by the present inventors and some others cited in the bibliography, it is possible to predict a high antitumor effect in the use of drugs that specifically achieve the inhibition of Rac activity. Of particular relevance are data showing, in this sense, the use of bacterial exoenzymes, such as Clostridium botulinum C3, which block the activity proteins of Rho family, because they include a ADP-Ribose in a protein aminoacidic residue that is essential for performing its GTPase function (Boquet P: Bacterial toxins inhibiting or activating small GTP-binding proteins. Ann N Y Acad Sci 1999, 886:83-90).

In scientific literature there are various studies about the use of compounds with GTPase inhibitory activity, using as target the nucleotide bond site (Yuan Gao, J. Bradley Dickerson, Fukun Guo, Jie Zheng and Yi Zheng: Rational design and characterization of a Rac GTPase-specific small molecule inhibitor. PNAS 2004, 101: 7618-7623).

In addition, the use of enzymes as biocatalysts has become of great importance in preparing organic compounds. Biocatalysis can be used for regioselectively or enantioselectively preparing compounds that could not be prepared by conventional chemical methods; this methodology allows carrying out different chemical reactions without the need of going through steps of protection and deprotection. Additionally, the high selectivity of biotransformations leads to the preparation of products in high yields without the formation of byproducts, which sometimes are very difficult to remove. The advantages of this technique in the preparation of pharmaceuticals have been made apparent in the last years because it allows for the resolution and synthesis of pharmaceuticals by means of processes not harmful to the environment. Moreover, the possibility of carrying out reactions with enzymes in organic media, as well as the wide substrate specificity shown by some of these biocatalysts, have led in pharmacy in the last years, to an increase in the number of patent application related to this methodology, displacing conventional chemical methods, specially those using heavy metals.

Although biocatalysis shows huge advantages over chemical catalysis, nowadays is considered of great importance for preparing “fine chemicals” to obtain a suitable combination of both processes.

The obtaining of compounds capable of specifically inhibiting Rho-GTPases activity offers a specific alternative in cancer therapy, taking into account the great evidence that relates this molecule family to tumoral progression.

Therefore, present invention relates to new antitumor agents the action of which interferes with the signaling pathways usually involved in tumor development processes. More specifically, the agents of the invention block the activation of the monomeric GTPases of the Rho-GTPase family. The activated GTP union site is proposed as new therapeutic target, using analogous to nucleoside with affinity for said site. In addition, the invention relates to new compounds with inhibitory activity of a Rho-GTPase cell protein, to the processes for obtaining thereof, to the pharmaceutical compositions comprising the same, and to their use in treating a condition mediated by a Rho-GTPase protein.

### BRIEF DESCRIPTION OF THE DRAWINGS

FIG. 1 shows Rac (Rac-GTP) activation levels as a response to treatment with Nucl 5 analog. A—Rac-GTP pull-down assay. The cells were treated with Nucl 5 increasing doses from 1 to 50 μM for 24 hs in the presence of 5% FBS. B—Densitometric analysis of Rac-GTP levels.

### BRIEF DESCRIPTION OF THE INVENTION

The present invention relates to a compound having inhibitory activity on a Rho-GTPase cell protein, the compound having general formula I

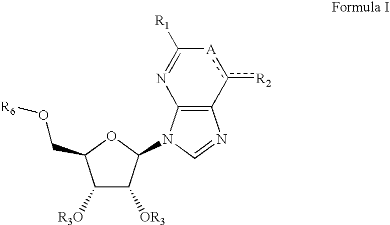

wherein
A is selected from N and N—H
R₁ is selected from H and NHR₃,
R₂ is selected from NHR₄, OR₄, 0 and halogen,
R₃ is selected from H and —COR₅,
R₄ is selected from H, C₁-C₆ alkyl and a substituted or unsubstituted phenyl,
R₅ is selected from C₁-C₁₂ alkyl and a substituted or unsubstituted phenyl,
R₆ is selected from H, —COR₅, —CO₂R₅, —PR₇R₈ and —PR₇R₈OPR₇R₈R₈R₇
R₇ is selected from O and S,
R₈ is selected from H, OR₄ and OSATE (—OCH₂CH₂SCOR₅), and
where [symbol] represents a single bond or a double bond, provided that when one of them is a double bond the other one is a single bond, and pharmaceutically acceptable salts and derivatives thereof.

In particular, the invention relates to a compound having inhibitory activity on a Rho-GTPase cell protein, the compound having general formula II

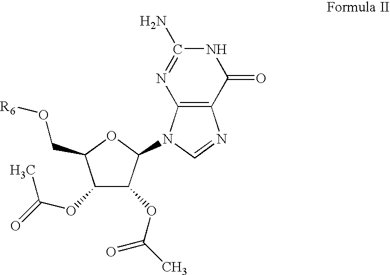

wherein R₆ has the above mentioned definition. Preferably, R₆ is selected from —COCH₃, —CO-phenyl and —COC₅H₁₁.

According to another embodiment, the invention refers to a compound having inhibitory activity on a Rho-GTPase cell protein, the compound having general formula III

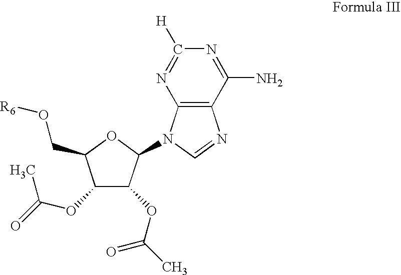

wherein R₆ is —COCH₃.

Further, yet another particular embodiment of the invention refers to a compound having inhibitory activity on a Rho-GTPase cell protein, the compound having general formula IV

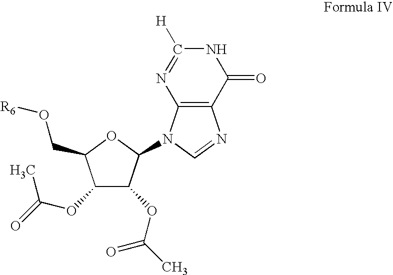

wherein R₆ is selected from H and —COCH₃.

Furthermore, particularly, a compound having inhibitory activity on a Rho-GTPase cell protein of the invention is selected from any one of the following compounds 1 to 8:

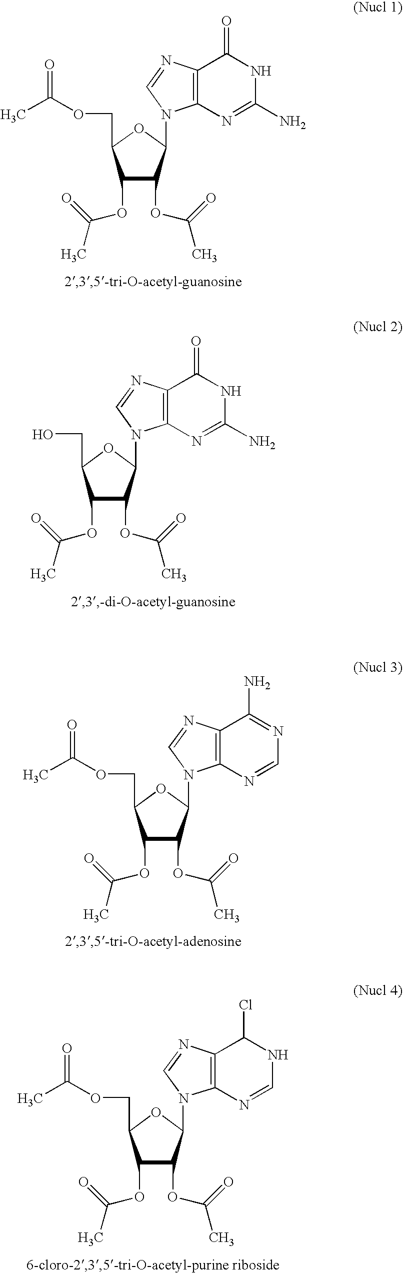

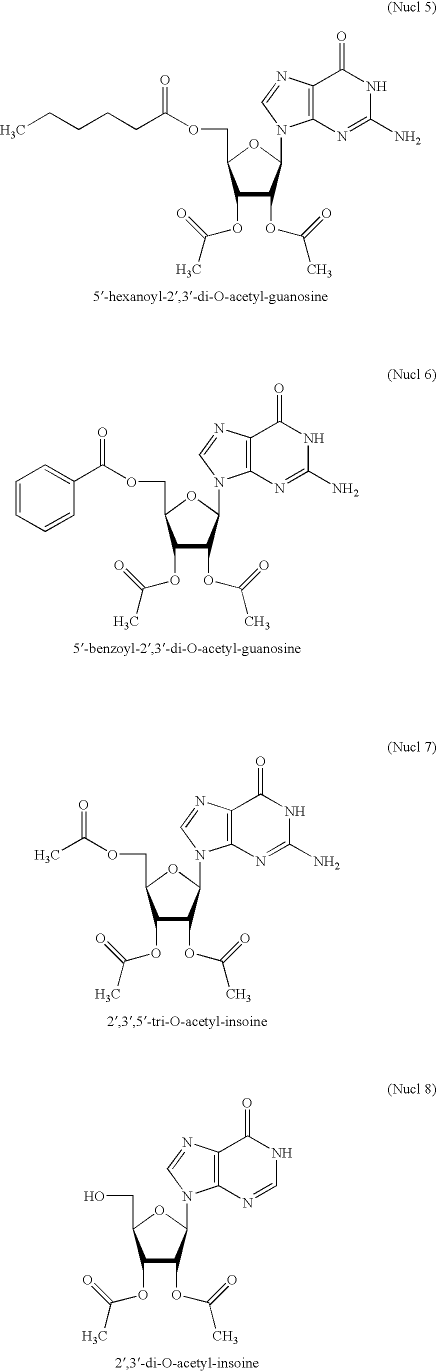

### DETAILED DESCRIPTION OF THE INVENTION

The present invention refers to a compound with inhibitory activity on a Rho-GTPase cell protein, the compound having the general formula I:

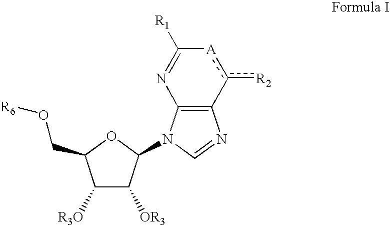

wherein A is selected from N and N—H;
R₁ is selected from H and NHR₃,
R₂ is selected from NHR₄, OR₄, O and halogen,
R₃ is selected from H and —COR₅,
R₄ is selected from H, a C₁-C₆ alkyl and a substituted or unsubstituted phenyl,
R₅ is selected from a C₁-C₁₂ alkyl and a substituted or unsubstituted phenyl,
R₆ is selected from H, —COR₅, —CO₂R₅, —PR₇R₈, —PR₇R₈OPR₇R₈R₈R₇
R₇ is selected from O and S,
R₈ is selected from H, OR₄ and OSATE (—OCH₂CH₂SCOR₅), and
wherein [symbol] represents a single bond or a double bond, provided that when one of them is a double bond the other one is a single bond, and pharmaceutically acceptable salts and derivatives thereof.

Besides, a particular embodiment of the invention refers to a compound with inhibitory activity on a Rho-GTPase cell protein, the compound having the general formula II:

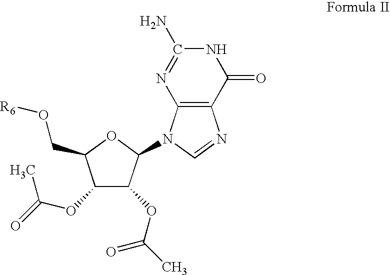

wherein R₆ has the same meaning as given above. Preferably, R₆ is selected from —COCH₃, H, —CO-phenyl and —COC₅H₁₁.

It is another particular embodiment of the invention, a compound with inhibitory activity on a Rho-GTPase cell protein, having the formula III:

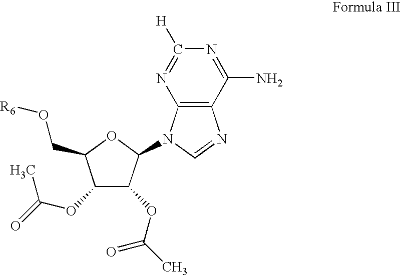

wherein R₆ is —COCH₃.

Besides, the invention particularly refers to a compound with inhibitory activity on a Rho-GTPase cell protein, the compound having the formula IV:

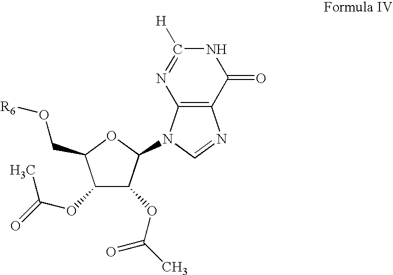

wherein R₆ is selected from H, —COR₅, —CO₂R₅, —PR₇R₈, —PR₇R₈OPR₇R₈R₈R₇, wherein R₇ and R₈ have the meaning given above. Preferably, R₆ is selected from H and —COCH₃.

It is another object of the invention, a pharmaceutical composition which comprises at least a compound with inhibitory activity on a Rho-GTPase cell protein, the compound having formula I, and a pharmaceutically acceptable carrier. In particular, it is another object of the invention, a pharmaceutical composition which comprises a compound with inhibitory activity on a Rho-GTPase cell protein, the compound having formula I, and pharmaceutically acceptable excipients. The person skilled in the art will know the most suitable excipients to be chosen for the composition of the invention, according to the selected way of administration.

Preferably, the pharmaceutical composition of the invention is an antitumoral composition.

In a particular way, the pharmaceutical composition of the invention is conceived to be administered orally, parenterally or transdermally. Particularly, said composition is in the form of a liquid, suspension, tablet, capsule, pill, injectable solution or transdermal patch. It is another particular embodiment of the invention, a pharmaceutical composition for the controlled release of at least one compound with inhibitory activity on a Rho-GTPase cell protein, the compound having the formula I. The person skilled in the art will know how to formulate the composition of the invention according to the selected way of administration.

Preferably, the pharmaceutical composition according to the invention comprises one or more compounds selected from 2′,3′,5′-tri-O-acetyl-guanosine, 2′,3′-di-O-acetyl-guanosine, 2′,3′,5′-tri-O-acetyl-adenosine, 6-chloro-2′,3′,5′-tri-O-acetyl-purine riboside, 5′hexanoyl-2′,3′-di-O-acetyl-guanosine, 5′-benzoyl-2′,3′-di-O-acetyl-guanosine, 2′,3′,5′-tri-O-acetyl-inosine and 2′,3′-di-O-acetyl-inosine. The composition of the invention may also comprise the compound of formula I in combination with other therapeutically active substances, which along with the compounds of the invention provide a synergistic or addition therapeutical effect, such as, for example, gemcitabine, capecitabine, decitabine, paclitaxel and docetaxel.

In another particular embodiment of the invention, the pharmaceutical composition comprises the therapeutically active substance/s encapsulated within liposomes or microspheres. The person skilled in the art will establish, considering the available literature related with this type of encapsulation, the necessary parameters and excipients.

In addition, it is another object of the invention, a method for the treatment of a Rho-GTPase cell protein mediated condition, which comprises administering to a patient in need thereof, a safe and effective amount of at least a compound of formula I. In particular, said Rho-GTPase cell protein is Rac1 and preferably said condition is abnormal cell proliferation or cancer cell proliferation. Besides, in a particular embodiment, the condition is selected form leukemia, prostate cancer, ovarian cancer, pancreas cancer, lung cancer, breast cancer, liver cancer, head or neck cancer, bladder cancer, non-Hodgkin's lymphomas and melanoma.

Contemplated within the scope of the invention, there is also a process for obtaining a compound of formula I, wherein said compound is selected from 2′,3′,5′-tri-O-acetyl-guanosine, 2′,3′,5′-tri-O-acetyl-inosine and 2′,3′,5′-tri-O-acetyl-adenosine, the process comprising reacting the corresponding free nucleoside with an acylating agent in the presence of acetonitrile, triethylamine and catalytic amounts of dimethylaminopyridine.

Also within the scope of the invention, there is contemplated a process for obtaining a compound of formula I, wherein said compound is selected from 2′,3′-di-O-acetyl-guanosine and 2′,3′-di-O-acetyl-inosine, the process comprising reacting the corresponding triacetylated nucleoside with ethanol in the presence of CAL B enzyme (Candida antarctica lipase B).

It is also comprised within the scope of the invention, a process for obtaining a compound of formula I, wherein said compound is selected from 5′-hexanoyl-2′,3′-di-O-acetyl-guanosine and 5′-benzoyl-2′,3′-di-O-acetyl-guanosine, which comprises reacting 2′,3′-di-O-acetyl-guanosine and the corresponding acylating agent with catalytic amounts of triethylamine, acetonitrile and dimethylaminopyridine.

In a particular embodiment of the invention, the process for obtaining the compounds of formula I uses biocatalysts instead of the catalysts used in conventional chemical reactions. Thus, technological and commercial advantages in terms of speed, costs and simplicity of manufacture over other pharmaceuticals of similar action, are generated.

### EXAMPLES

### Example 1

### Synthesis of the Compounds According to the Invention

For obtaining the 2′,3′,5′-tri-O-acetyl-guanosine (Nucl 1), 2′,3′,5′-tri-O-acetyl-inosine (Nucl 7), 2′,3′,5′-tri-O-acetyl-adenosine (Nucl 3) and 6-chloro-2′,3′,5′-tri-O-acetyl-purine riboside (Nucl 4), the corresponding free nucleoside was used as a starting material (10 mmol guanosine, inosine, adenosine or 6-chloro-purine riboside) and they were reacted in the presence of an acylating agent (40 mmol acetic anhydride), using acetonitrile as the solvent (125 ml) and catalytic amounts of triethylamine (40 mmol) and dimethylaminopyridine (105 mg). After 2 hours, the reaction was stopped by adding methanol (15 ml). The obtained mixture was evaporated in vacuo using a rotary evaporator. The purification step was performed by column chromatography using dichloromethane/methanol as the elution solvent. The structure of the compound was confirmed by nuclear magnetic resonance.

For obtaining the compounds 2′,3′-di-O-acetyl-guanosine (Nucl 2) and 2′,3′-di-O-acetyl-inosine (Nucl 8), the corresponding triacetylated nucleoside synthesized according to the above disclosure was used as a starting material (1 mmol 2′,3′,5′-tri-O-acetyl-guanosine or 2′,3′,5′-tri-O-acetyl-inosine, respectively) and it was resuspended in ethanol (1 mol), which acts as nucleophile and reaction solvent, in the presence of 300 mg of the CAL B enzyme (Candida Antarctica lipase B). After the appropriate reaction time, the reaction was stopped by filtration the enzyme. The obtained mixture was evaporated in vacuo using a rotary evaporator. The purification step was performed by column chromatography using dichloromethane/methanol (98/2 v/v) as the elution solvent. The structure of the compound was confirmed by nuclear magnetic resonance.

For obtaining the 5′-hexanoyl-2′,3′-di-O-acetyl-guanosine (Nucl 5) and 5′-benzoyl-2′,3′-di-O-acetyl-guanosine (Nucl 6), the 2′,3′-di-O-acetyl-guanosine (1 mmol), synthesized according to the above disclosure was used as a starting material, and it was reacted in the presence of the corresponding acylating agent (1 mmol hexanoic anhydride or benzoyl chloride) using acetonitrile as the solvent (15 ml), triethylamine (1.5 mmol) and dimethylaminopyridine in catalytic amounts (10 mg). After 2 hours, the reaction was stopped by adding methanol (5 ml). The obtained mixture was evaporated in the presence of vacuum using a rotary evaporator. The purification step was performed by column chromatography using dichloromethane/methanol (98/2 v/v) as the elution solvent. The structure of the compound was confirmed by nuclear magnetic resonance.

### Characterization of the Compounds Nucl 1, Nucl 2, Nucl 4, Nucl 5, Nucl 7 and Nucl 8 by Means of ¹³C-NMR Technique

| Compound | C2 | C4 | C5 | C6 | C8 | C1′ | C2′ | C3′ | C4′ | C5′ | carbonyl | acyls |
|---|---|---|---|---|---|---|---|---|---|---|---|---|
| Nucl 1 | 153.2 | 151.3 | 116.6 | 156.7 | 136.0 | 87.3 | 72.7 | 71.4 | 79.5 | 63.1 | 169.2 | 20.2 |
| | | | | | | | | | | | 169.6 | 20.4 |
| | | | | | | | | | | | 170.1 | 20.5 |
| Nucl 2 | 154.0 | 151.3 | 116.6 | 156.7 | 135.3 | 83.6 | 72.7 | 71.4 | 74.6 | 61.0 | 169.2 | 20.2 |
| | | | | | | | | | | | 169.6 | 20.5 |
| Nucl 4 | 152.2 | 151.3 | 132.2 | 149.0 | 144.2 | 85.8 | 72.3 | 70.0 | 79.6 | 61.61 | 169.4 | 20.2 |
| | | | | | | | | | | | 169.6 | 20.4 |
| | | | | | | | | | | | 170.3 | 20.6 |
| Nucl 5 | 153.1 | 151.4 | 116.6 | 156.9 | 136.0 | 87.0 | 72.7 | 71.5 | 79.6 | 63.5 | 169.2 | 13.6 |
| | | | | | | | | | | | 172.1 | 13.9 |
| | | | | | | | | | | | 173.2 | 21.7 |
| | | | | | | | | | | | | 23.8 |
| Nucl 7 | 145.3 | 148.8 | 125.4 | 159.1 | 138.4 | 86.4 | 73.3 | 70.4 | 80.6 | 62.9 | 172.1 | 20.4 |
| | | | | | | | | | | | 172.3 | 20.5 |
| | | | | | | | | | | | 173.2 | 20.6 |
| Nucl 8 | 146.5 | 148.0 | 126.1 | 158.3 | 139.9 | 86.2 | 73.3 | 72.3 | Not | 62.4 | 172.0 | 20.4 |
| | | | | | | | | | observed | | 171.9 | 20.6 |

Characterization of Compounds Nucl 3 and Nucl 6 by their Melting Points

Nucl 3: m.p. 70-72° C.

Nucl 6: m.p. 78-80° C.

### Example 2

The “Molecular Docking” has been a major focus of interest in the last years. In general, the current “docking” software, such as DOCK, Auto Dock, FlexX, GOLD, etc., are capable of predicting the protein-ligand complexes structure with good efficiency and velocity. When it is used previously to the experimental screening, it is considered as a computational filter that enables the reduction of experimentation times and costs.

The present inventors have optimized a group of computational tools with the objective of designing molecules with high affinity for the selected molecular target, i.e. the Rac1 GTPase.

The docking is mainly based on the calculus of free energy values as a product of the interaction between the flexible ligand and the amino acids belonging to the binding site of the protein, apart from taking into consideration the steric and spatial distribution parameters of the atoms which are part of the molecule. In general, the less the free energy values obtained, the higher the protein-ligand affinity. From the above, it follows that, from the thermodynamic point of view, the interaction between said molecules would be more favored if the free energy values obtained are lower. For the obtained energy values to have a predictive value, they must be compared with energy values for known ligand-protein complexes for the selected target. In present case, the energy values obtained for the compounds of interest were compared with the free energy values calculated for the Rac1-GTPβS complex. This complex is thought to be optimal since GTPβS is a structural analog to the natural ligand of the protein (GGTP-GDP). Besides, the stronger data in this sense is that this complex co-crystallizes along with it. The structure of the target protein is obtained from the crystallography published in the Protein Data Bank.

TABLE 1
Docking of the 8 molecules with the binding site of
the Rac nucleosides. (Table with binding energy and docking
energy values)

| | Binding | Docking |
|---|---|---|
| Compound | Energy | Energy |
| 2′,3′,5′-tri-O-acetyl-guanosine | −9.06 | −10.9 |
| (Nucl 1) | | |
| 2′,3′-di-O-acetyl-guanosine | −9.74 | −11.2 |
| (Nucl 2) | | |
| 2′,3′,5′-tri-O-acetyl-adenosine | −10.8 | −13.2 |
| (Nucl 3) | | |
| 6-chloro-2′,3′,5′-tri-O-acetyl- | N/A | N/A |
| purine riboside (Nucl 4) | | |
| 5′-hexanoyl-2′,3′-di-O-acetyl- | −10.71 | −12.62 |
| guanosine (Nucl 5) | | |
| 5′-benzoyl-2′,3′-di-O-acetyl- | −8.05 | −9.5 |
| guanosine (Nucl 6) | | |
| 2′,3′,5′-tri-O-acetyl-inosine | −6.22 | −8.71 |
| (Nucl 7) | | |
| 2′,3′-di-O-acetyl-inosine (Nucl 8) | −9.02 | −10.4 |

### Example 2a

### Inhibition of the Rac Activation Levels in Response to Nucl5

This assay consists in the determination of inhibition power of the compound 5′-hexanoyl-2′,3′-di-O-acetyl-guanosine (Nucl 5) over the intracellular active Rac levels (Rac-GTP). For determining the levels of Rac-GTP, the “Pull-Down” assay was used, which is based in the conformation bond of Rac-GTP to the p21 domain of PAK1 protein, which is the direct effector of Rac-GTP (Wang H and Kazanietz M G: Chimaerins, novel “non-PKC” phorbol ester receptor, associate with Tmp21-I (p23). Evidence for a novel anchoring mechanism involving the chimaerin C1 domain. J. Biol. Chem. 2002, 277: 4541-4550). In order to achieve said objective, cells were seeded in 6-well cell culture plaques and were kept in absence of FBS for 24 hours (starvation). Afterwards, they were stimulated for 10 minutes with EGF (100 ng/ml), washed with phosphate buffered saline (PBS) at low temperature and lysed in a buffer containing 8 μg of the fusion protein GST-PBD, 20 mM Tris-HCl, pH 7.5, 1 mM DTT, 5 mM MgCl₂, 150 mM NaCl, 0.5% NP-40, 5 mM β-glicerofosfato and protease inhibitors (5 μg/ml 4-(2-aminoethyl)bencenesulfonyl fluoride, 5 μg/ml leupeptin, 5 μg/ml aprotinin and 1 μg/ml pepstatin A). The lysates were centrifuged at 14,000×g (4° C., 10 min) and then incubated with glutation-Sefarosa 4B microspheres (Amersham Pharmacia) at 4° C. for 2 hours. After extensive washings, the microspheres were boiled for 5 minutes in loading buffer. The samples were separated in a 12% SDS-polyacrilamide gel and electro-transferred to a PVDF membrane for its ulterior “Western Blot” analysis using an anti-Rac1 antibody (Sigma). The total Rac levels were analyzed in a similar way from aliquots taken from the cell lysates.

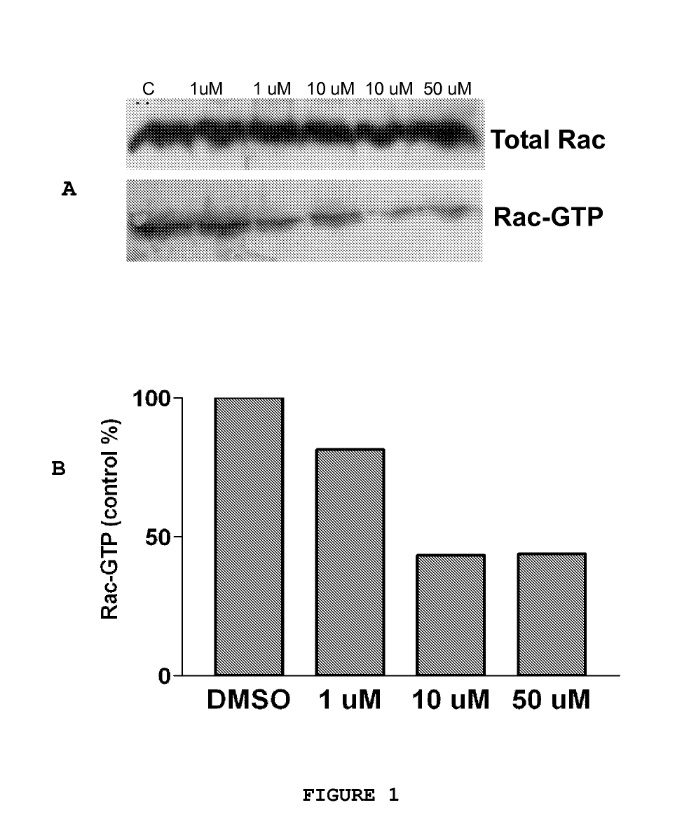

The results of the pull-down experiment showed a marked dose-dependant reduction of the Rac activation levels in response to the cell treatment with the compound, manifesting its highest inhibitory capacity up from approximately 10 μM (FIG. 1). The Rac (Rac-GTP) activation levels are depicted in FIG. 1 as a response to the treatment with analog Nucl 5. A. Pull-down test on Rac-GTP. The cells were treated with increasing doses of Nucl 5 from 1 to 50 μM for 24 hs in the presence of 5% FBS. B Densitometric analysis of the Rac-GTP levels.

### Example 2b

### Antiproliferative Effect of the Compounds Nucl 1, Nucl 4 and Nucl 5 on Breast Carcinoma F3II Cells

The antiproliferative effect of the above mentioned compounds on breast carcinoma F3II cells is disclosed in the present example. The synthesized compounds are purine nucleoside derivatives which present chemical modifications in either the ribose and/or in the base. These modifications mainly consisted in acetylations of positions 2′-3′ of the ribose, varying the number of carbon atoms of the acyl substituents in position 5′. The cells were treated for 72 hours in the presence of fetal bovine serum 10% with different doses of the compounds, with the aim of determining the inhibitory concentration 50% (IC50). The cell growth was estimated by toluidine test, for which the cell monolayers were fixed with formalin (Formol 10%) and stained with toluidine blue 0.5%. Subsequently, the stained cells were thoroughly washed with PBS and solubilized with 1% SDS. Finally, the number of cells was estimated by measuring the absorbance values at 595 nm. The IC50 values of the compounds Nucl 1, Nucl 4 and Nucl 5, both for cells in the exponential growth phase and for quiescent cells, are depicted in Table 2.

TABLE 2
IC50 values of the analogs tested in F3II cells at
exponential and stationary growth phases.

| | |
|---|---|
| Growing Cells | Quiescent Cells |

| Compounds | IC50 48 hs | IC50 72 hs | IC50 24 hs |
|---|---|---|---|
| Nucl 1 | 26 μM | 68 μM | >100 μM |
| Nucl 4 | 60 μM | 56 μM | >100 μM |
| Nucl 5 | NT | 73 μM | >100 μM |

In addition, the effect of the analogs showing antiproliferative effect on quiescent cells, was studied in order to discriminate between an antitumoral effect and an unspecific cytotoxic effect. For this purpose, the semiconfluent cell monolayers were incubated for 24 hours in the absence of FBS with the analogs Nucl 1, Nucl 4 and Nucl 5, at a concentration of 100 μM. Subsequently, the cells were fixed and stained with toluidine in order to determine the number of cells. Simultaneously, the cytotoxic effect was determined by the MTT assay. In none of the cases a cytotoxic effect was observed as a result of the treatment with the tested compounds, thus indicating that the effect observed on the tumor cells growth is not due to a direct cytotoxic effect.

### Example 3

### Antimigration Effect of the Compounds of the Invention on Breast Carcinoma Cells

The present assay consists in the determination of the antimigration effect of the selected compounds on the breast carcinoma cells migration. Cell motility is a key process in the invasion and tumor metastasis processes and it is closely regulated by the Rho-GTPases family, particularly by Rac.

Tumor cell migration in vitro was measured by the “monolayer wound” assay described previously (Alonso D F, Farias E F, Urtreger A, Ladeda V, Vidal M C and Bal de Kier Joffé E: Characterization of F3II, a mammary sarcomatoid carcinoma cell line originated from a mouse adenocarcinoma. J. Surgh. Oncol 1996, 62: 288-297). 0.5 mm width lines were made in confluent monolayers. After washing with PBS, the tumor cells were incubated overnight in MEM with 10% FBS and the monolayers were fixed and stained with methylene blue. The number of cells that moved along the free space were counted by using a 0.36 mm² graded grid, using a magnification of 100×, in 3 independent fields per each line.

Doses from 1 to 50 μM of the compounds were assessed, thereby finding a saturation of the antimigration effect in the 3 compounds studied, starting from 10 μM, obtaining an approximate inhibition of 30 to 50% respectively.

TABLE 3
Effect of compounds Nucl 1, Nucl 4 and Nucl 5 over
breast carcinoma F3II migration.

Cell Migration

| | N^{o} of | | |
|---|---|---|---|
| | migrating | | |
| Compounds (10 μM) | cells | SD | Inhibition % |
| Control | 144 | 52.8 | — |
| Nucl 1 | 97.7* | 33.52 | 32.2 |
| Nucl 4 | 85.5** | 42.70 | 40.6 |
| Nucl 5 | 70.7** | 22.7 | 51.0 |

*p < 0.05,
**p < 0.01, ANOVA test contrasted with Dunnet's Test.

### Example 4

### Effect of Compound Nucl 5 on Tumor Growth In Vivo Of a Murine Breast Carcinoma Line

Present assay is a study of the in vivo antitumor effect of compound Nucl 5. BALB/c female mice were subcutaneously inoculated with 2×10⁵ F3II cells. The animals were treated with daily intraperitoneal doses of 1, 5 y 10 mg/kg/day of Nucl 5. The inoculated mice were monitored for parameters of toxicity associated to the treatment, such as daily water and food intake and body weight. Besides, the antitumor effect of the compounds was assessed from the latency and tumor incidence assessment, tumor growth rate and survival.

As to toxicity parameters, no effects on the treated batches were observed at 1 and 5 mg/kg/day strengths, noting a slight increase in water intake at 10 mg/kg/day dose. It is probable that said slight increase is associated to a diuretic effect, induced by the solute concentration increase. Some symptoms of irritability were also observed in the animals treated with this Nucl 5 strength.

Table 4 shows the incidence, tumor latency and survival data. Treatment with the analogue Nucl 5 had no noticeable effects on incidence and tumor latency. However, it had a significant effect on the survival of treated animals. Accordingly, a clear increase in survival of mice treated with all tested doses could be observed. As to the tumor progression, no substantial difference was observed in the initial kinetics of subcutaneous tumor growth. Nevertheless, the tumor volume on day 22 at 5 mg/kg/day dose showed a significant reduction (329.4±50.7 vs. 133.8±45.1*p<0.05 ANOVA contrasted with Dunnet's test)

TABLE 4
Incidence, Latency and Survival values of mice
inoculated with 2 × 10⁵ F3II cells and treated with
intraperitoneal daily doses of Nucl 5 of 1, 5 y 10 mg/kg/day.

| | Tumor | Tumor | |
|---|---|---|---|
| Treatments | Incidence | Latency | Survival Day 50 |
| Physiological | 100% | 4-8 days | 0-5 (0%) |
| Soln. | | | |
| 1 mg/kg/day | 100% | 4-8 days | 5-5 (100%) |
| 5 mg/kg/day | 100% | 4-6 days | 5-5 (100%) |
| 10 mg/kg/day | 100% | ~5 days | 4-5 (80%) |

### Example 5

### Antimetastatic Effect of Compound Nucl 5 on a Breast Carcinoma Line

Present assay is a study of the antimetastatic effect of compound Nucl 5 on a highly invasive and metastatic breast carcinoma. Thus, on the so-called day 0 of the experiment, BALB/c female mice were inoculated intravenously with 2×10⁵ F3II cells. On day 21 the animals were sacrificed by cervical dislocation and submitted to histopathological analysis. In order to investigate the presence of lung metastasis, the same were removed and fixed in Bouin's solution, and the number of superficial metastatic nodules was determined under dissecting microscope.

For the study of the effect of the compound on the metastatic colonization, daily intraperitoneal doses of 5 mg/kg/day were administered, from day −1 to day +3. In another experiment, the effect of the compound on the metastatic nodules formation and growth was studied, by administering daily intraperitoneal doses from day −1 up to the end of the experiment (day +21). The obtained results are listed in Table 5. The daily treatment with the compound at a 5 mg/kg/day dose from the previous day up to 3 days after the intravenous inoculation of tumor cells, significantly reduced metastatic nodule formation in lungs. Besides, the daily administration up to the end of the experiment produced a similar antimetastatic action.

TABLE 5
Effect of Nucl 5 compound on the metastatic
colonization of F3II breast carcinoma cells. Each experimental
group comprises 10 animals.

| | Nodules per animal |
|---|---|
| Treatment | (media ± SEM) |
| control | 15 ± 1.63 |
| Nucl 5 days −1 + 3 | 9.2 ± 1.36* |
| Nucl 5 days −1 + 21 | 9.4 ± 1.6* |

*p < 0.05. Anova contrasted with Dunnet's Test.

### Example 6

The following example shows the preparation of representative pharmaceutical compositions comprising a compound of formula I according to the invention:

Injectable formulation

| | | |
|---|---|---|
| Compound of formula I | 0.01 | g |
| Propyleneglycol | 20 | g |
| Polyethyleneglycol 400 | 20 | g |
| Tween 80 | 1 | g |
| Saline 0.9% | q.s. 100 | ml |

Oral formulation

| | | |
|---|---|---|
| Compound of formula I | 0.01 | g |
| Pregelatinized starch | 74.8 | g |
| Microcrystalline cellulose, | 20 | g |
| Magnesium stearate | 0.2 | g |
| Ethylcellulose | | |
| Triacetin. | | |

1-27. (canceled)

28. A compound having formula II 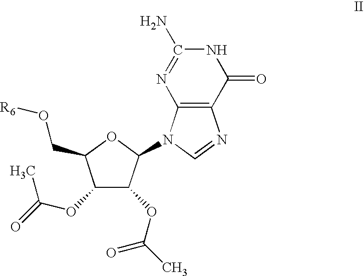
wherein
R₆ is selected from —CO-phenyl and —COC₅H₁₁, and pharmaceutically acceptable salts thereof.

29. A pharmaceutical composition comprising at least one compound according to claim 28 and a pharmaceutically acceptable carrier.

30. The pharmaceutical composition according to claim 29, comprising a compound according to claim 28 and pharmaceutically acceptable excipients.

31. The pharmaceutical composition according to claim 29, suitable for being administered orally, parenterally or transdermally.

32. The pharmaceutical composition according to claim 31, in the form of a liquid, suspension, tablet, capsule, pill, injectable solution or transdermal patch.

33. The pharmaceutical composition according to claim 29, wherein said composition is a controlled release composition.

34. The pharmaceutical composition according to claim 29, comprising at least one compound selected from 5′-hexanoyl-2′,3′-di-O-acetyl-guanosine and 5′-benzoyl-2′, 3′-di-O-acetyl-guanosine.

35. The pharmaceutical composition according to claim 29 comprising another therapeutically active substance.

36. The pharmaceutical composition according to claim 29, wherein the therapeutically active substance(s) is(are) encapsulated within liposomes or microspheres.

37. A pharmaceutical composition according to claim 29, wherein said composition is an antitumor composition.

38. A process for obtaining a compound according to claim 28, wherein said compound is selected from 5′-O-hexanoyl-2′,3′-di-O-acetyl-guanosine and 5′-O-benzoyl-2′,3′-di-O-acetyl-guanosine, said process comprising reacting 2′,3′-di-O-acetyl-guanosine and the corresponding acylating agent in the presence of triethylamine, acetonitrile and catalytic amounts of dimethylaminopyridine.

39. A method for treating a condition mediated by a Rho-GTPase cell protein, which comprises administering to a patient in need thereof a safe and effective amount of at least a compound of formula I: 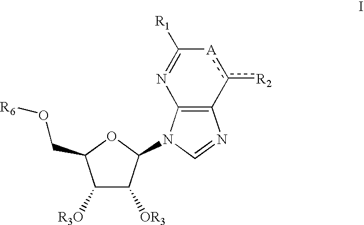
wherein A is selected from N and N—H
R₁ is selected from H and NHR₃,
R₂ is selected from NHR₄, OR₄, O and halogen,
R₃ is selected from H and —COR₅,
R₄ is selected from H, a C₁-C₆ alkyl and a substituted or unsubstituted phenyl,
R₅ is selected from a C₁-C₁₂ alkyl and a substituted or unsubstituted phenyl,
R₆ is selected from H, —COR₅, —CO₂R₅, —PR₇R₈ and —PR₇R₈OPR₇R₈R₈R₇
R₇ is selected from O and S,
R₈ is selected from H, OR₄ and OSATE (—OCH₂CH₂SCOR₅), and
wherein each [symbol] represents a single bond or a double bond, provided that when one of them is a double bond the other one is a single bond, and pharmaceutically acceptable salts thereof.

40. The method according to claim 39, which comprises administering to a patient in need thereof a safe and effective amount of at least a compound of formula II: 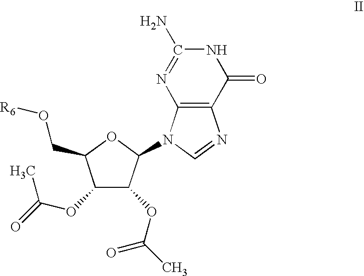
wherein
R₆ is selected from —CO-phenyl and —COC₅H₁₁, and pharmaceutically acceptable salts thereof.

41. The method according to claim 39, wherein said Rho-GTPase cell protein is Rac1.

42. The method according to claim 39, wherein the condition is leukemia, prostate cancer, ovary cancer, pancreas cancer, lung cancer, breast cancer, liver cancer, head or neck cancer, bladder cancer, non-Hodgkin's lymphomas and melanoma.

43. The method according to claim 39, wherein the condition is abnormal cell proliferation.

44. The method according to claim 39, wherein the condition is cancer cell proliferation.
